(19) United States
(12) Patent Application Publication
(10) Pub. No.: US 2022/0202998 A1
(43) Pub. Date: Jun. 30, 2022

(54) MEDICAL DEVICE AND METHOD FOR PRODUCING MEDICAL DEVICE

(71) Applicant: TERUMO KABUSHIKI KAISHA, Tokyo (JP)

(72) Inventor: Naoto TAKEMURA, Atsugi-shi (JP)

(73) Assignee: TERUMO KABUSHIKI KAISHA, Tokyo (JP)

(21) Appl. No.: 17/695,790

(22) Filed: Mar. 15, 2022

Related U.S. Application Data
(63) Continuation of application No. PCT/JP2020/030403, filed on Aug. 7, 2020.

(30) Foreign Application Priority Data
Sep. 17, 2019 (JP) 2019-168674

(51) Int. Cl. A61L 29/06; A61L 29/08; A61L 31/06; A61L 31/10; A61L 31/14

(52) U.S. Cl. CPC A61L 29/06; A61L 29/085; C08L 83/04; A61L 31/10; A61L 31/14; A61L 31/06

(57) ABSTRACT

A medical device including: a substrate having a mottled structure containing silicone on a surface of the substrate; and a coating layer containing a polyalkylene glycol on a surface of the substrate.

### CROSS-REFERENCE TO RELATED APPLICATIONS

This application is a bypass continuation of PCT Application No. PCT/JP2020/030403, filed on Aug. 7, 2020, which claims priority to Japanese Application No. 2019-168674, filed on Sep. 17, 2019. The contents of these applications are hereby incorporated by reference in their entireties.

### BACKGROUND

The present disclosure relates to a medical device and a method for producing the medical device.

Medical devices inserted into a living body, such as a catheter and an indwelling needle, are used for infusion, blood transfusion, and the like. As such a medical device, a medical device whose surface is treated with silicone in order to impart lubricity and reduce friction at the time of puncture is known. For example, JP S61-35870 B discloses an injection needle surface-treated with a composition containing, as a main component, a reaction product between a reaction product of an amino group-containing silane and an epoxy group-containing silane, and a polydiorganosiloxane containing a silanol group.

### SUMMARY

The injection needle described in JP S61-35870 B certainly has an excellent puncture property due to its surface coated with silicone.

However, when the surface of a medical device (for example, an indwelling catheter) indwelled in a blood vessel is coated with the composition described in JP S61-35870 B, there is a problem that antithrombogenicity may be insufficient depending on the indwelling time (for example, 24 hours or more).

Therefore, certain embodiments of the present invention have been developed in view of the above circumstances, and an object of certain embodiments of the present invention is to provide a medical device having an excellent sliding property (in particular, a puncture property) and exhibiting excellent antithrombogenicity.

The present inventor has conducted intensive studies in order to solve the above problem. As a result, the present inventor has found that the above problem is solved by a medical device including a substrate having a mottled structure containing silicone on a surface of the substrate and a coating layer containing a polyalkylene glycol on a surface of the substrate.

### BRIEF DESCRIPTION OF THE DRAWINGS

FIG. 1 is a schematic view of blood circulation experiments used in Examples and Comparative Examples;

FIG. 2 is a laser micrograph showing the surface structure of a substrate having a mottled structure on a surface thereof, prepared in Examples;

FIG. 3 is a laser micrograph for measuring a surface structure; and

FIG. 4 is a laser micrograph for showing a method for measuring a surface structure.

### DETAILED DESCRIPTION

Hereinafter, embodiments of the present invention will be described. However, the present invention is not limited only to the following embodiments.

In the present specification, “X to Y” indicating a range means “X or more and Y or less.” In addition, unless otherwise specified, operations and measurements of physical properties and the like are performed under the conditions of a room temperature of 25±1° C./a and a relative humidity of 40 to 50% RH.

<Medical Device>

According to one embodiment of the present invention, a medical device includes: a substrate having a mottled structure containing silicone on a surface thereof; and a coating layer containing a polyalkylene glycol on a surface of the substrate. When the medical device includes a substrate having a mottled structure containing silicone on a surface thereof and a coating layer containing a polyalkylene glycol on a surface of the substrate, the medical device can have an excellent sliding property (in particular, a puncture property) and exhibit excellent antithrombogenicity.

In the present specification, the “surface” of the substrate refers to a surface on a side with which blood or the like comes into contact when a medical device is used and a surface portion of a cavity in the substrate. For example, when the medical device is an indwelling catheter, the surface means an outer surface and/or an inner surface.

[Substrate Having Mottled Structure Containing Silicone on Surface]

The substrate according to the present embodiment has a mottled structure containing silicone on a surface thereof.

(Substrate)

The material of the substrate main body (the material of the substrate before formation of the mottled structure) is not particularly limited. Examples thereof include various polymer materials including: polyolefins or modified polyolefins such as polyethylenes, polypropylenes, and ethylene-α-olefin copolymers; polyamides; polyimides; polyurethanes; polyesters such as polyethylene terephthalate (PET), polybutylene terephthalate (PBT), polycyclohexane terephthalate, and polyethylene-2,6-naphthalate; polyvinyl chloride; polyvinylidene chloride (PVDC); fluororesins such as polytetrafluoroethylene (PTFE) and ethylene-tetrafluoroethylene copolymers (ETFEs), metals, ceramics, carbon, and composite materials thereof. The polymer material may be subjected to a stretching treatment (for example, ePTFE).

The shape of the substrate is appropriately selected according to the application of the medical device or the like, and may be, for example, a tubular shape, a sheet shape, a rod shape, or the like. The form of the substrate is not limited to a formed body prepared using the above-described material alone, and a blend formed product, an alloyed formed product, a multilayered formed product, or the like can be used. The substrate may be a single layer or may be laminated. At this time, when the substrates are laminated, the substrates of the respective layers may be the same or different.

(Mottled Structure)

In the invention according to the present embodiment, the surface of the substrate constituting the medical device is configured with a mottled structure. The surface of the substrate is formed in an uneven shape. That is, in an embodiment, the mottled structure includes a convex portion formed on the surface and a concave portion formed on the surface. The convex portion formed on the surface contains silicone. The concave portion formed on the surface is not substantially covered, and the surface of the substrate is exposed. The convex portion has a portion in which a plurality of granular projections are randomly connected to form a meandering linear body in a plan view. The concave portion surrounds the convex portion in a plan view so that the linear bodies of the convex portion are arranged in a relatively dispersed manner. Such a state in which the convex portion and the concave portion are irregular is referred to as a mottled pattern. The mottled structure can be confirmed by observation with a laser microscope (objective lens: 150 times).

FIG. 2 is an image showing the surface structure of a substrate having a mottled structure on a surface thereof, prepared in Examples. The dark color portion corresponds to the convex portion, and the light color portion corresponds to the concave portion. The dark color portion corresponding to the convex portion extends in a random direction. The light color concave portion surrounds the convex portion and is continuous with another concave portion. The surface of the substrate is in a state in which the convex portion and the concave portion are irregular as described above. A structure in which the dark color portion is connected to the other portion at a plurality of sites can also be expressed as a mesh structure.

It can be confirmed by elemental analysis that silicone is contained in the convex portion.

When the surface of the substrate has a mottled structure containing silicone and a coating layer containing a polyalkylene glycol on the surface of the substrate, the medical device of the present embodiment can have an excellent sliding property (in particular, a puncture property) and exhibit excellent antithrombogenicity.

It is considered that antithrombogenicity and a sliding property are exhibited by forming a mottled pattern with an appropriate size and distribution as a mottled structure containing silicone on the surface of the substrate. The mechanism by which silicone forms a mottled pattern is considered to be related to the interaction of a polyalkylene glycol as a second component such as polyethylene glycol with a substrate and silicone.

In a preferred embodiment, the average convex width (width of the convex portion) of the mottled structure is preferably in a range of 0.1 to 10 μm, and more preferably 0.5 to 3 μm from the viewpoint that the effect of the present invention can be further exhibited. The average concave width (width of the concave portion) of the mottled structure is preferably in a range of 0.1 to 10 μm, and more preferably 0.5 to 3 μm.

In a preferred embodiment, the ratio of the average convex width to the average concave width (average convex width/average concave width) of the mottled structure is preferably in a range of 0.1 to 5, more preferably in a range of 0.3 to 3, and still more preferably in a range of 0.4 to 2 from the viewpoint that the effect of the present invention can be further exhibited.

The average convex width and the average concave width of the mottled structure can be measured by the following method.

First, the surface of the medical device having a mottled structure is observed with a laser microscope (VKX-100, manufactured by Keyence Corporation, objective lens: 150 times, monitor magnification: 3,000 times), and an image is captured. The captured image is analyzed with image analysis software. Specifically, an optional straight line (first straight line) is drawn on the image, and a second straight line orthogonal to the first straight line is drawn. A portion of the convex portion intersecting each straight line is defined as a convex width, and a portion of the concave portion intersecting each straight line is defined as a concave width. The average convex width is obtained by arithmetically averaging the convex widths obtained from at least nine points. The average concave width is obtained by taking the concave widths from at least nine points and arithmetically averaging the obtained concave widths. The convex width a and the concave width b are measured values in the X direction, and the convex width a′ and the concave width b′ are measured values in the Y direction. When b is smaller than b′, a and b are adopted, and when b is larger than b′, a′ and b′ are adopted.

Silicone

The silicone according to the present embodiment is not particularly limited, and biocompatible silicone can be appropriately used. As the silicone, a crosslinked silicone is preferably used from the viewpoint of form stability.

The crosslinked silicone is a silicone containing a three-dimensional bond. Specific examples of the crosslinked silicone include a reaction product between a reaction product of an amino group-containing silane and an epoxy group-containing silane, and a polydiorganosiloxane containing a silanol group described in JP S61-35870 B or JP S62-52796 B, and a copolymer of aminoalkylsiloxane and dimethylsiloxane described in JP S46-3627 B.

Examples of the amino group-containing silane include γ-aminopropyltriethoxysilane, γ-aminopropylmethyldiethoxysilane, N-(β-aminoethyl)aminomethyltrimethoxysilane, γ-M-aminoethyl)aminopropyltrimethoxysilane, γ-(N-(β-aminoethyl)amino)propylmethyldimethoxysilane, N-M-aminoethyl)aminomethyltributoxysilane, and γ-(N-(β-(N-(β-aminoethyl)amino)ethyl)amino)propyltrimethoxysilane.

Examples of the epoxy group-containing silane include γ-glycidoxypropyltrimethoxysilane, γ-glycidoxypropylmethyldimethoxysilane, β-(3,4-epoxycyclohexyl)ethyltrimethoxysilane, β-(3,4-epoxycyclohexyl)ethylmethyldimethoxysilane, β-(3,4-epoxycyclohexyl)ethyltriethoxysilane, and β-(3,4-epoxycyclohexyl)ethylmethyldiethoxysilane.

The polydiorganosiloxane containing a silanol group has at least one silanol group in one molecule. The viscosity of the polydiorganosiloxane containing a silanol group is in a range of 0.00002 to 1 m²/s, and preferably in a range of 0.0001 to 0.1 m²/s at 25° C. When the viscosity is 0.00002 m²/s or more, a sufficient puncture property can be obtained. When the viscosity is 1 m²/s or less, handling before curing is facilitated. Examples of the organic group bonded to the silicon atom of the silanol group include an alkyl group such as a methyl group, a phenyl group, and a vinyl group. From the viewpoint of ease of synthesis of polydiorganosiloxane, the organic group is preferably a methyl group or a phenyl group, and more preferably a methyl group. Specific examples of the polydiorganosiloxane containing a silanol group include polydimethylsiloxane in which one terminal is blocked with a silanol group and the other terminal is blocked with a trimethylsilyl group, polydimethylsiloxane in which both terminals are blocked with a silanol group, and polymethylphenylsiloxane in which both terminals are blocked with a silanol group.

The reaction product of an amino group-containing silane and an epoxy group-containing silane can be obtained by reacting the amino group-containing silane and the epoxy group-containing silane by heating with stirring.

For the reaction ratio between the amino group-containing silane and the epoxy group-containing silane, the ratio of the epoxy group-containing silane is in a range of 0.5 to 3.0 mol, and preferably 0.75 to 1.5 mol with respect to 1 mol of the amino group-containing silane.

The reaction product between the reaction product (A component) of the amino group-containing silane and the epoxy group-containing silane, and the polydiorganosiloxane (B component) containing a silanol group can be obtained by reacting the A component and the B component while heating using a solvent as necessary. For the blending ratio between the A component and the B component, the blending ratio of the A component is in a range of 0.1 to 10 mass %, and the blending ratio of the B component is in a range of 90 to 99.9 mass %, with respect to the total of the A component and the B component. Preferably, the blending ratio of the A component is in a range of 1 to 5 mass %, and the blending ratio of the B component is in a range of 95 to 99 mass %.

As the crosslinked silicone, a commercially available product can be used. Examples of a commercially available product that can be used include MDX4-4159 (manufactured by The Dow Chemical Company).

Other Components

The mottled structure according to the present embodiment can further contain components other than silicone. Examples of the other component include hydrophilic polymers such as polymethoxyethyl acrylate (PMEA) that can be dissolved in a solvent common to polyalkylene glycol and silicone, and organic compounds having a pharmacological action or an antibacterial action.

In an embodiment, the mottled structure contains a polyalkylene glycol in addition to silicone. When the mottled structure contains a polyalkylene glycol, a sliding property, specifically, a puncture property can be improved. Polyalkylene glycol is eluted during puncture, and this makes it possible to reduce the resistance. In addition, a polyalkylene glycol having a higher molecular weight (weight average molecular weight) can improve a sliding property.

The polyalkylene glycol is preferably selected from polyethylene glycol, polypropylene glycol, and polybutylene glycol, and more preferably polyethylene glycol, from the viewpoint of improving the puncture property.

The weight average molecular weight of the polyalkylene glycol is, for example, in a range of 100 to 10,000,000, preferably 200 to 4,000,000, and more preferably 400 to 500,000. As the weight average molecular weight, a value measured by gel permeation chromatography (Gel Permeation Chromatography, GPC) using polystyrene as a standard substance and tetrahydrofuran (THF) as a mobile phase is adopted.

[Coating Layer Containing Polyalkylene Glycol]

The medical device of the present embodiment has a coating layer containing a polyalkylene glycol on a surface of the substrate. This makes it possible to further improve the puncture property among the effects of the mottled structure on the substrate surface.

The coating layer may have a form in which the entire surface of the substrate is completely covered with the coating layer, or a form in which only a part of the surface of the substrate is covered with the coating layer, that is, a form in which the coating layer is attached to only a part of the surface of the substrate.

The polyalkylene glycol is preferably selected from polyethylene glycol, polypropylene glycol, and polybutylene glycol, and more preferably polyethylene glycol, from the viewpoint of improving the puncture property.

The weight average molecular weight of the polyalkylene glycol is, for example, in a range of 400 to 10,000,000, preferably 1,000 to 4,000,000, and more preferably 2,000 to 500,000. As the weight average molecular weight of the polyalkylene glycol increases, the puncture property can be further improved.

As the weight average molecular weight, a value measured by gel permeation chromatography (Gel Permeation Chromatography, GPC) using polystyrene as a standard substance and tetrahydrofuran (THF) as a mobile phase is adopted.

The coating layer may be formed of only a polyalkylene glycol, or may contain a component other than polyalkylene glycol as long as the effect of the present invention is not impaired. Examples of the component other than polyalkylene glycol include glycerin and water-soluble compounds having a pharmacological action or an antibacterial action.

When the coating layer contains a component other than polyalkylene glycol, the content of the component is, for example, more than 0 mass % and 50 mass % or less with respect to the total mass of the coating layer.

[Medical Device]

Examples of the medical device of the present embodiment include a device used in contact with body fluid, blood, or the like. As described above, when the surface of the medical device has the mottled structure containing silicone and the coating layer containing a polyalkylene glycol on a surface of the substrate, the medical device can have an excellent sliding property (in particular, a puncture property) and exhibit excellent antithrombogenicity. Therefore, the medical device of the present embodiment may be used in any application as long as a puncture property and/or antithrombogenicity are required. Examples include a catheter, a sheath, a cannula, a needle, a three-way stopcock, and a guide wire. Other examples include a blood circuit, an artificial dialyzer, an artificial (auxiliary) heart, an artificial lung, an indwelling needle, an artificial kidney, and a stent. In the case of a medical device inserted into or placed in a body cavity such as a blood vessel, the above-described structure can be provided on the outer surface of at least a part of the device in order to improve a sliding property when the medical device comes into contact with the body cavity. In the case of a medical device in which a second device is inserted into the internal space thereof, such as a catheter or a sheath, the above structure can be provided on at least a part of the surface of the internal space in order to improve a sliding property when the second device is inserted. In particular, the medical device of the present embodiment can achieve both a sliding property, particularly a puncture property, and antithrombogenicity, and thus is suitably used as an indwelling catheter.

<Method for Producing Medical Device>

According to another embodiment of the present invention, a method for producing a medical device includes: applying a mixed solution containing silicone and a polyalkylene glycol to a substrate to prepare a substrate having a mottled structure containing silicone on a surface thereof; and applying a solution containing a polyalkylene glycol to the substrate having a mottled structure containing silicone on a surface thereof to form a coating layer.

[Step of Preparing Substrate Having Mottled Structure Containing Silicone on Surface]

In this step, a mixed solution containing silicone and a polyalkylene glycol is applied to a substrate.

The silicone, the polyalkylene glycol, and the substrate are similar to those in the form of the substrate having a mottled structure containing silicone on a surface thereof in the medical device, described above, and thus the description thereof is omitted.

The method for preparing the mixed solution is not particularly limited, and for example, the mixed solution can be prepared by dissolving silicone and a polyalkylene glycol in a solvent. The solvent is not particularly limited as long as it can dissolve silicone and a polyalkylene glycol. For example, as a solvent for the crosslinked silicone and the polyalkylene glycol, dichloropentafluoropropane, methylene chloride, hydrochlorofluoroolefin, trans-1,2-dichloroethylene, chloroform, or the like can be used.

The concentration of the silicone in the mixed solution is not particularly limited as long as it is a concentration that allows formation of a mottled structure on the surface of the substrate, and is, for example, in a range of 0.1 to 20 v/v %, and preferably 1 to 10 v/v %.

The concentration of the polyalkylene glycol in the mixed solution is not particularly limited as long as it is a concentration that allows formation of a mottled structure on the surface of the substrate, and is, for example, in a range of 0.1 w/v % or more and less than 2.0 w/v %, and preferably 0.1 to 1.0 w/v %.

In an embodiment, the mixed solution according to the production method of the present embodiment contains an amount of silicone in a range of 1 to 10 v/v % and an amount of polyalkylene glycol in a range of 0.1 to 1.0 w/v %.

By appropriately adjusting the types of silicone and polyalkylene glycol to be used and the concentrations of silicone and polyalkylene glycol in the mixed solution, the average convex width and the average concave width of the mottled structure formed on the surface of the medical device can be adjusted.

The method for applying the mixed solution to the substrate is not particularly limited, and conventionally known methods such as a coating and printing method, an immersion method (dipping method, dip coating method), a spraying method, a spin coating method, and a mixed solution impregnated sponge coating method can be used.

In a preferred embodiment of the present embodiment, the method for applying the mixed solution to the substrate is an immersion method (dipping method). The immersion temperature is not particularly limited, and is, for example, in a range of 10 to 50° C., and preferably 15 to 40° C. The immersion time is not particularly limited, and is, for example, in a range of 10 seconds to 30 minutes.

In the case of forming a mottled structure on a thin and narrow inner surface of a catheter, a guide wire, an injection needle, or the like, the substrate is immersed in the mixed solution, and degassing may be performed by reducing the pressure in the system. By performing degassing under reduced pressure, the solution is quickly penetrated into the narrow inner surface, and whereby the formation of mottled structure can be promoted.

After the substrate is immersed in the mixed solution, the substrate is taken out and subjected to a drying treatment. The speed at which the substrate is pulled up is not particularly limited, and is, for example, in a range of 5 to 50 mm/sec. The drying condition (temperature, time, etc.) is not particularly limited as long as it is a condition under which the mottled structure can be formed on the surface of the substrate. Specifically, the drying temperature is preferably in a range of 20 to 150° C. The drying time is preferably in a range of 20 minutes to 2 hours, and more preferably 30 minutes to 1 hour.

The pressure condition during drying is not limited at all, and the drying may be performed under normal pressure (atmospheric pressure), or may be performed under compression or decompression.

As the drying means (apparatus), for example, an oven, a vacuum dryer, or the like can be used, but in the case of natural drying, the drying means (apparatus) is not particularly necessary.

By the above method, a substrate having a mottled structure containing silicone on a surface thereof can be produced.

[Step of Forming Coating Layer]

In this step, a solution containing a polyalkylene glycol is applied to the substrate having a mottled structure containing silicone on a surface thereof.

The polyalkylene glycol is similar to that in the form of the coating layer containing the polyalkylene glycol of the medical device, and thus the description thereof is omitted.

The method for preparing the solution is not particularly limited, and for example, the solution can be prepared by dissolving a polyalkylene glycol in a solvent. The solvent is not particularly limited as long as it can dissolve a polyalkylene glycol. When the polyalkylene glycol is polyethylene glycol, for example, water, an alcohol such as methanol, ethanol, and isopropyl alcohol, acetone, tetrahydrofuran, dichloropentafluoropropane, methylene chloride, hydrochlorofluoroolefin, trans-1,2-dichloroethylene, and chloroform can be used as the solvent.

The concentration of the polyalkylene glycol in the solution is appropriately adjusted according to the weight average molecular weight of the polyalkylene glycol to be used, and is, for example, 0.1 to 40 w/v %.

When the coating layer contains a component other than the polyalkylene glycol, the content of the component in the solution can be appropriately adjusted so as to be, for example, more than 0 mass % and 50 mass % or less with respect to the total mass of the coating layer.

The method for applying the solution to the substrate having the mottled structure containing silicone on a surface thereof is not particularly limited, and conventionally known methods such as a coating and printing method, an immersion method (dipping method, dip coating method), a spraying method, a spin coating method, and a mixed solution impregnated sponge coating method can be used similarly to the step of producing the substrate having a mottled structure containing silicone on a surface thereof.

In a preferred embodiment of the present embodiment, the method for applying the solution to the substrate having a mottled structure containing silicone on a surface thereof is an immersion method (dipping method). The immersion temperature is not particularly limited, and is, for example, in a range of 10 to 50° C., and preferably 15 to 40° C. The immersion time is not particularly limited, and is, for example, in a range of 10 seconds to 30 minutes.

After immersing the substrate having a mottled structure containing silicone on a surface thereof in the solution, the substrate is taken out and subjected to a drying treatment. The speed at which the substrate is pulled up is not particularly limited, and is, for example, in a range of 5 to 50 mm/sec. The drying condition (temperature, time, etc.) is not particularly limited as long as it is a condition under which the coating layer can be formed on the surface of the substrate. Specifically, the drying temperature is preferably in a range of 20 to 150° C. The drying time is preferably in a range of 20 minutes to 2 hours, and more preferably 30 minutes to 1 hour.

The pressure condition during drying is not limited at all, and the drying may be performed under normal pressure (atmospheric pressure), or may be performed under compression or decompression.

As the drying means (apparatus), for example, an oven, a vacuum dryer, or the like can be used, but in the case of natural drying, the drying means (apparatus) is not particularly necessary.

By the above method, a coating layer containing a polyalkylene glycol can be formed.

### EXAMPLES

The effects of certain embodiments of the present invention will be described using the following Examples and Comparative Examples. However, the technical scope of the present invention is not limited only to the following examples. Unless otherwise specified, the operation was performed at room temperature (25° C.). In addition, unless otherwise specified, “%” and “part” mean “wt %” and “part by weight”, respectively.

(Preparation of Catheter Substrate)

Extrusion molding was performed using a polyurethane resin (manufactured by Nippon Miractran Co, Ltd.), and then annealing was performed at 100° C. for 1 hour to prepare a catheter substrate.

(Preparation of Substrate Having Mottled Structure on Surface)

Polyethylene glycol (PEG) (weight average molecular weight: 4,000) and a crosslinked silicone produced based on Coating Agent Preparation Example 1 described in JP S61-35870 A were dissolved at 0.5 w/v % and 3 v/v %, respectively, in ASAHIKLIN AK225 (dichloropentafluoropropane; AGC Inc.) to prepare a mixed solution. The catheter substrate prepared above was immersed in the mixed solution for 10 seconds using a ROBO Cylinder manufactured by IAI Corporation, pulled up at a speed of 5 mm/sec, and dried at 60° C. for 30 minutes. Thereafter, the substrate was immersed in RO water to prepare a substrate having a mottled structure on a surface thereof. When the prepared substrate was confirmed using a laser microscope (objective lens: 150 times), a mottled structure was formed on the surface (FIG. 2).

### Example 1

Polyethylene glycol (PEG) (weight average molecular weight: 4,000) was dissolved at 30 w/v % in ASAHIKLIN AK225 to prepare a PEG solution. The substrate having the mottled structure on the surface prepared above was immersed in the PEG solution using a ROBO Cylinder manufactured by IAI Corporation, pulled up at a speed of 5 mm/sec, and dried at 60° C. for 30 minutes to produce a catheter.

### Example 2

Polyethylene glycol (PEG) (weight average molecular weight: 50,000) was dissolved at 1 w/v % in ASAHIKLIN AK225 to prepare a PEG solution. The substrate having the mottled structure on the surface prepared above was immersed in the PEG solution using a ROBO Cylinder manufactured by IAI Corporation, pulled up at a speed of 5 mm/sec, and dried at 60° C. for 30 minutes to produce a catheter.

### Example 3

Polyethylene glycol (PEG) (weight average molecular weight: 350,000) was dissolved at 0.5 w/v % in ASAHIKLIN AK225 to prepare a PEG solution. The substrate having the mottled structure on the surface prepared above was immersed in the PEG solution using a ROBO Cylinder manufactured by IAI Corporation, pulled up at a speed of 5 mm/sec, and dried at 60° C. for 30 minutes to produce a catheter.

### Comparative Example 1

Polyethylene glycol (PEG) (weight average molecular weight: 4,000) was dissolved at 30 w/v % in ASAHIKLIN AK225 to prepare a PEG solution. The catheter substrate prepared as described above was immersed in the PEG solution using a ROBO Cylinder manufactured by IAI Corporation, pulled up at a speed of 5 mm/sec, and dried at 60° C. for 30 minutes to produce a catheter.

### Comparative Example 2

Polyethylene glycol (PEG) (weight average molecular weight: 50,000) was dissolved at 1 w/v % in ASAHIKLIN AK225 to prepare a PEG solution. The catheter substrate prepared as described above was immersed in the PEG solution using a ROBO Cylinder manufactured by IAI Corporation, pulled up at a speed of 5 mm/sec, and dried at 60° C. for 30 minutes to produce a catheter.

### Comparative Example 3

Polyethylene glycol (PEG) (weight average molecular weight: 350,000) was dissolved at 0.5 w/v % in ASAHIKLIN AK225 to prepare a PEG solution. The catheter substrate prepared as described above was immersed in the PEG solution using a ROBO Cylinder manufactured by IAI Corporation, pulled up at a speed of 5 mm/sec, and dried at 60° C. for 30 minutes to produce a catheter.

<Evaluation>

[Puncture Resistance Evaluation]

The puncture resistance (trunk portion resistance) was measured for the catheters of Examples 1 to 3 and the comparative catheters of Comparative Examples 1 to 3. Specifically, an inner needle was incorporated into a catheter having an inner diameter of 0.8 mm and an outer diameter of 1.1 mm. A polyethylene film having a thickness of 50 μm was punctured with water dripping at an angle of 90 degrees and a speed of 30 ram/min using a small desktop tester EZ-1 manufactured by Shimadzu Corporation. A maximum resistance value after passage of 10 mm from the needle tip was measured. The results are shown in Table 1.

[Antithrombogenicity Evaluation]

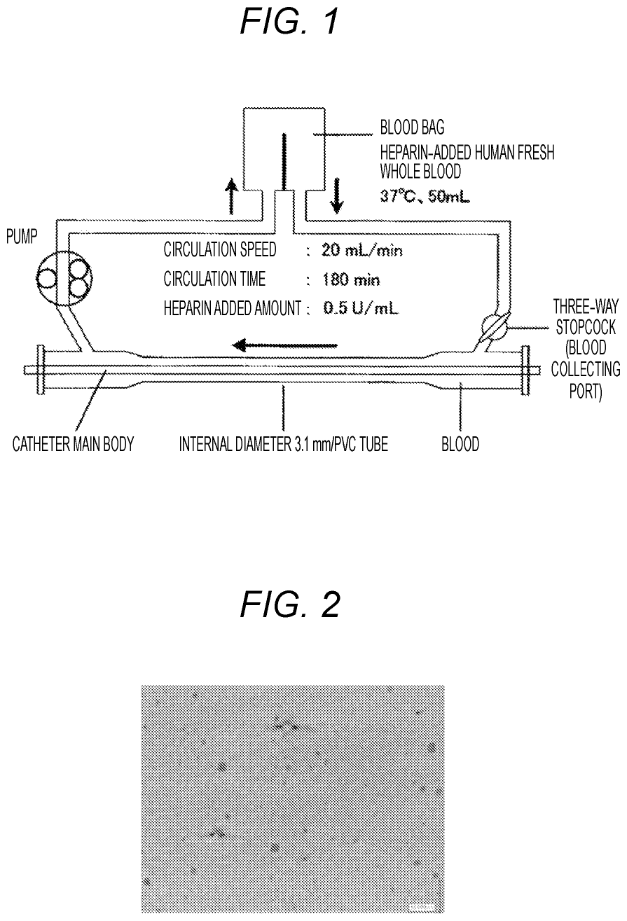

For the catheters of Examples 1 to 3 and the comparative catheters of Comparative Examples 1 to 3, a blood circulation experiment was performed for 3 hours in the system shown in FIG. 1. After circulation, the production amount of thrombin-antithrombin complex (TAT) was measured. The production amount of TAT was measured by an EIA method. The results are shown in Table 1.

[Measurement of Mottled Structure]

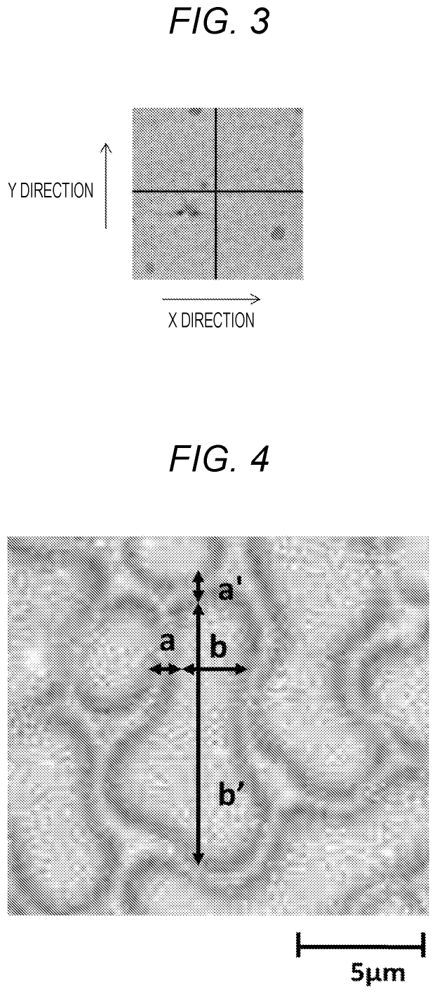

The substrate having a mottled structure on the surface prepared above was observed with a laser microscope (objective lens: 150 times), and an image was captured. The captured image was analyzed with image analysis software. The convex width a of the mottled structure and the concave width b of the mottled structure were each measured at nine points in the X direction (catheter axial direction), and similarly, also in the Y direction orthogonal to the X direction, the convex width a′ of the mottled structure and the concave width b′ of the mottled structure were each measured at nine points (see FIGS. 3 and 4). The average convex width and the average concave width were calculated by arithmetically averaging the obtained convex widths and concave widths. The results are shown in Table 1.

TABLE 1

| | Substrate | | | | | | | | | |
|---|---|---|---|---|---|---|---|---|---|---|
| | | | | Surface structure | | | Coating layer | | Evaluation | |
| | Mixed solution | | | | Mottled | | Solution | | | Puncture |
| | PEG | | | | structure | | PEG | | Antithrombogenicity | resistance |
| | | Weight | Crosslinked | | Average | Average | | Weight | evaluation | evaluation |
| | Concen- | average | silicone | | convex | concave | Concen- | average | TAT production | Trunk portion |
| | tration | molecular | Concentration | | width | width | tration | molecular | amount | resistance |
| | (w/v %) | weight | (v/v %) | Type | (μm) | (μm) | (w/v %) | weight | (ng/mL) | (N) |
| Example 1 | 0.5 | 4000 | 3.0 | Mottled | 1.84 | 1.01 | 30 | 4000 | 300 | 0.063 |
| Example 2 | 0.5 | 4000 | 3.0 | pattern | | | 1 | 50000 | 300 | 0.060 |
| Example 3 | 0.5 | 4000 | 3.0 | | | | 0.5 | 350000 | 300 | 0.059 |
| Comparative | — | | — | None | — | — | 30 | 4000 | 530 | 0.120 |
| Example 1 | | | | | | | | | | |
| Comparative | | | | | | | 1 | 50000 | 530 | 0.084 |
| Example 2 | | | | | | | | | | |
| Comparative | | | | | | | 0.5 | 350000 | 530 | 0.073 |
| Example 3 | | | | | | | | | | |

Table 1 shows that the catheters of the Examples have an excellent sliding property, specifically, a puncture property, and exhibit excellent antithrombogenicity as compared with the comparative catheters of the Comparative Examples.

What is claimed is:

1. A medical device comprising:
a substrate having a mottled structure containing silicone at a surface of the substrate; and
a coating layer containing a polyalkylene glycol on a surface of the substrate.

2. The medical device according to claim 1, wherein the polyalkylene glycol contained in the coating layer is polyethylene glycol.

3. The medical device according to claim 1, wherein the mottled structure further contains a polyalkylene glycol.

4. The medical device according to claim 2, wherein the mottled structure further contains a polyalkylene glycol.

5. The medical device according to claim 1, wherein the mottled structure includes a convex portion formed on a surface of the substrate and a concave portion formed on a surface of the substrate.

6. The medical device according to claim 2, wherein the mottled structure includes a convex portion formed on a surface of the substrate and a concave portion formed on a surface of the substrate.

7. The medical device according to claim 3, wherein the mottled structure includes a convex portion formed on a surface of the substrate and a concave portion formed on a surface of the substrate.

8. The medical device according to claim 4, wherein the mottled structure includes a convex portion formed on a surface of the substrate and a concave portion formed on a surface of the substrate.

9. The medical device according to claim 5, wherein an average width of the convex portion of the mottled structure is in a range of 0.1 to 10 μm, and an average width of the concave portion of the mottled structure is in a range of 0.1 to 10 μm.

10. The medical device according to claim 6, wherein an average width of the convex portion of the mottled structure is in a range of 0.1 to 10 μm, and an average width of the concave portion of the mottled structure is in a range of 0.1 to 10 μm.

11. The medical device according to claim 7, wherein an average width of the convex portion of the mottled structure is in a range of 0.1 to 10 μm, and an average width of the concave portion of the mottled structure is in a range of 0.1 to 10 μm.

12. The medical device according to claim 8, wherein an average width of the convex portion of the mottled structure is in a range of 0.1 to 10 μm, and an average width of the concave portion of the mottled structure is in a range of 0.1 to 10 μm.

13. A method for producing a medical device, the method comprising:
applying a mixed solution containing silicone and a polyalkylene glycol to a substrate to prepare a substrate having a mottled structure containing silicone at a surface of the substrate; and
applying a solution containing a polyalkylene glycol to the substrate having a mottled structure containing silicone on a surface of the substrate to form a coating layer.

14. The method according to claim 13, wherein the mixed solution contains 1 to 10 v/v % of silicone and 0.1 to 1.0 w/v % of a polyalkylene glycol.
